(19) United States
(12) Patent Application Publication
(10) Pub. No.: US 2025/0189440 A1
(43) Pub. Date: Jun. 12, 2025

(54) PULSE LASER SPECTROMETER

(71) Applicant: EMERSON PROCESS MANAGEMENT LIMITED, Leicestershire (GB)

(72) Inventors: David LOGIE, Sterling (GB); Giuseppe SMIRNE, Sterling (GB)

(21) Appl. No.: 18/975,916

(22) Filed: Dec. 10, 2024

(30) Foreign Application Priority Data
Dec. 11, 2023 (GB) 2318868.3

(51) Int. Cl. G01N 21/31; G01N 33/00

(52) U.S. Cl. CPC G01N 21/31; G01N 33/0027; G01N 2201/0612

(57) ABSTRACT

A method for sensing gases using a semiconductor diode laser, the method comprising: applying an electrical current pulse to a semiconductor diode laser thereby to cause the laser to produce a laser output pulse, wherein the application of the electrical pulse to the semiconductor diode laser causes an increase in temperature such that the produced laser output pulse comprises a continuous wavelength chirp over a wavelength range; providing the produced laser output pulse to the gas sample region, wherein at least part of the wavelength range of the wavelength chirp is used as a wavelength scan; and detecting optical output from the gas sample region, wherein the wavelength chirp is caused, at least in part, by the increase in temperature of the laser induced by the applied electrical pulse, wherein the electrical current pulse comprises an electrical pulse length, or duration, of at least 5 microseconds and/or wherein the length of the electrical current pulse causes the increase in temperature to slow over the length of the current pulse thereby causing the rate of change of the continuous wavelength chirp to continuously slow over the length of the pulse from a first initial rate to a second, slower rate, and wherein the detection and/or further sampling of the optical output is performed at the second, slower rate.

### CROSS-REFERENCE TO RELATED APPLICATION

This application claims the benefit of Great Britain Application No. 2318868.3 filed 11 Dec. 2023, the entire contents of which are incorporated herein by reference in their entirety.

### FIELD

The present invention relates to a semiconductor diode laser spectrometer arrangement and in particular an infrared semiconductor diode laser spectrometer.

### BACKGROUND

WO 03/087787 describes applying a current pulse with a short temporal length to a quantum cascade laser operating near room temperature to provide a narrow wavelength pulse. WO 03/087787 describes applying a series of substantially step function electrical pulses to a semiconductor laser to output one or more laser pulses each having a continuous wavelength chirp. WO 03/087787 describes applying pulses in the range 150 to 300 ns, preferably 200 to 300 ns to provide a tuning range of about 60 GHz. In WO 03/087787 the wavelength variation provided by the wavelength chirp itself is used to provide a wavelength scan and therefore there is no need to tune an effective emission linewidth (the up-chirp) across a spectral region, using, for example, by using a slow DC current ramp superimposed on the pulse train.

WO 03/087787 describes that, due to the controllable and predictable characteristics, the almost linear wavenumber chirp (as a function of time) allows the construction of a high speed, sub-microsecond, semiconductor diode laser absorption spectrometer. In that document, a pulse generator provides a plurality of pulses (rectangular) to an input of the laser. The generator provides a train of fixed amplitude sub-microsecond duration rectangular current drive pulses to cause a fast laser heating effect and hence a continuous wavelength up-chirp of the emitted semiconductor diode laser radiation at a rate in time. The laser heating caused by the current pulses is such that for each pulse emitted from the laser, the chirp is a continuous almost linear spectral variation from short to long wavelength (defined as a continuous spectral or wavelength scan).

### SUMMARY

In accordance with a first aspect, there is provided a method for sensing gases using a semiconductor diode laser, the method comprising: applying an electrical pulse to a semiconductor diode laser thereby to cause the laser to produce a laser output pulse, wherein the application of the electrical pulse to the semiconductor diode laser causes a change in temperature such that the produced laser output pulse comprises a continuous wavelength chirp over a wavelength range; providing the produced laser output pulse to the gas sample region, wherein at least part of the wavelength range of the wavelength chirp is used as a wavelength scan; and detecting optical output from the gas sample region, wherein the electrical current pulse comprises an electrical pulse length of at least 1 microsecond. The wavelength chirp may be caused, at least in part, by the increase in temperature of the laser induced by the applied electrical pulse. The electrical current pulse comprises an electrical pulse length, or duration, of at least 5 microseconds. The length of the electrical current pulse may cause the increase in temperature to slow over the length of the current pulse thereby causing the rate of change of the continuous wavelength chirp to continuously slow over the length of the pulse from a first initial rate to a second, slower rate, and wherein the detection and/or further sampling of the optical output is performed at the second, slower rate.

The change in temperature may comprise an increase in temperature. The application of the electrical current pulse to the semiconductor may provide a heating effect to the semiconductor laser. The method may comprises sensing a gas and/or sensing one or more compounds in a sample gas and/or one or more materials in a sample gas. The method may comprise identifying one or more compounds in a sample gas.

The method may comprises applying one or more electrical current pulses to a semiconductor diode laser thereby to cause a temperature change in the semiconductor laser. The laser may output a continuous wavelength chirp, wherein the continuous wavelength chirp is produced at least in part due to the temperature change of the semiconductor laser.

The increase in temperature may slow over the length of the current pulse thereby causing the rate of change of the continuous wavelength chirp to continuously slow over the length of the pulse from a first initial rate to a second, slower rate, and wherein the detection and/or further sampling of the optical output is performed at the second, slower rate. The increase in temperature may slow over the length of the current pulse thereby causing the rate of change of the continuous wavelength chirp to monotonically decrease over the length of the pulse.

Detecting the optical output may comprise producing an electrical detection signal using one or more photodetectors in response to laser light incident on a detector and performing a digitisation process on the electrical detection signal.

The length of the electrical pulse may be sufficiently long that the rate of change of the produced wavelength chirp slows over the produced wavelength chirp. The length of the electrical pulse may have a duration such that the rate of change of the produced wavelength chirp slows over the produced wavelength chirp. The length of the electrical pulse may have a duration such that the produced wavelength chirp has a first rate of change at a first time and a second, lower rate of change at a second time. The rate of change of the produced wavelength chirp may continuously slow from a first rate of change to a second rate of change over the length of the produced continuous wavelength chirp. The rate of change may slow from a first rate of change to a second rate of change. The rate of change may be monotonically decreasing.

The length of the electrical pulse may be sufficiently long that the rate of change of the produced wavelength chirp slows by at least 25%, optionally 50%, further optionally 75% over the produced wavelength chirp.

The temperature may change over the pulse length. The rate of change in temperature may decrease as the pulse length gets longer. The rate of change of the produced wavelength chirp may slow over the duration of the produced wavelength chirp. The temperature may change over the pulse length due to a self-heating effect of the laser during production of the pulse. The rate of change in temperature may decrease as the pulse length gets longer. The rate of change of the produced wavelength chirp may slow over the duration of the produced wavelength chirp.

The change in temperature may comprise an increase in temperature. The size of the increase may be dependent on the laser. The change in temperature may comprise a change from a first temperature to a second temperature. The increase in temperature may comprise an increase in the range of 50° C. to 80° C. The increase in temperature may comprise an increase in the range of 1 degree C. to 100 degree C. The increase in temperature of the laser may slow over the electrical pulse length.

The current pulse may be sufficiently long that the increase in temperature of the laser slows over the electrical pulse length causing the chirp to be non-linear and/or the rate of change of the produced wavelength chirp to slow over the produced wavelength chirp

The chirp may be represented by a polynomial and/or exponential function and/or other non-linear function and/or the current pulse may comprises a length or duration such that the continuous wavelength chirp is non-linear in time.

The wavelength scan may comprise a portion of the wavelength chirp. The wavelength scan may comprise or correspond to a detection time window. The wavelength scan and/or detection window may comprise up to 75%, up to 50%, up to 25%, up to 10% of the wavelength chirp.

The method may comprise detecting light and/or sampling during a portion of the wavelength chirp comprising a slower rate of change.

The method may comprise detecting light and/or sampling during a portion of the wavelength chirp comprising a rate of change in the range 0.1 to 2 cm 1 per microsecond.

The one or more properties of the wavelength chirp may be dependent on at least the temperature of the laser and/or wherein the rate of change of the temperature decreases as the electrical pulse is applied over the electrical pulse length and/or wherein the temperature change may comprise an increase up to a maximum temperature, wherein the maximum temperature is dependent on one or more of an ambient temperature and/or a cooling effect.

The pulse may be such that a frequency of a corresponding etalon trace decreases over time.

The wavelength chirp may comprise of or be characterised by a chirp rate that decreases over the wavelength chirp. The wavelength chirp may comprise a portion that occurs over or defines a detection time window. The portion may be characterized by a chirp rate in a desired range.

The method may comprise detecting output from the sensing region and/or processing detection signals during the detection time window. The chirp may be non-linear.

The detection window may start at a time beyond 1 microsecond from the start of the pulse. The detection window may starts at a time above 2 microseconds, optionally 5 microseconds, optionally above 10 microseconds from the start of the pulse.

The wavelength chirp may comprise a chirp rate that decreases over the wavelength chirp, wherein the wavelength chirp comprises a portion characterized by a chirp rate in a desired range, wherein the portion occurs during at least a detection time window wherein the method comprises detecting output from the sensing region and/or processing detection signals during at least the detection time window. The portion may comprise a useable portion. The portion may have a duration that is at least the size of the detection time window. The portion may define the detection time window. The portion may have a duration that the size of the detection time window. The desired chirp rate may be in the range 0.1 to 2 cm⁻¹ per microsecond.

The start of the detection time window may be fixed and/or may be synchronized with the laser output.

The method may comprise detecting and/or sampling emitted light over the length of the chirp and discarding data collected outside the detection time window and/or processing only data collected during the detection time window.

The electrical pulse length may be between 1 microsecond and 200 microseconds, optionally between 100 microseconds and 10 milliseconds, further optionally between 100 microseconds and 1 millisecond.

The electrical current pulse may comprises an electrical pulse length optionally at least 5 microsecond, optionally at least 10 microseconds, further optionally between 5 microseconds and 1 millisecond, further optionally between 10 microseconds and 1 millisecond.

The first rate may be above 2 cm⁻¹ per microsecond and the second rate may be below 2 cm⁻¹ per microsecond, optionally wherein the first rate is above 2 cm⁻¹ per microsecond and the second rate is in a range of 0.1 to 2 cm⁻¹ per microsecond, further optionally wherein the second rate is below 0.1 cm⁻¹ per microsecond.

The first rate may be above a pre-determine value and the second rate may be below the pre-determined value. The pre-determined value may be dependent on the one or parameters, for example, the laser system.

The electrical pulse may comprise a current pulse.

The chirp rate may slow by up to 25%, optionally 50%, optionally over 100%.

The detection and digitising circuitry may comprise a photodetector having a bandwidth in the range 1 to 10 MHz and digitizing circuitry having a bandwidth in the range 1 to 10 MHz. The detection and digitising circuitry may comprise a photodetector and digitizing circuitry having a bandwidth in the range 1 to 20 MHz

The detection of the optical input may comprise detection and/or digitising by a photodetector and digitisation circuitry, wherein the detection and/or digitisation is performed when the wavelength chirp is in the range of range of 0.1 to 2 cm⁻¹ per microsecond.

The laser light may be directed to the gas analysis region using reflective optical components. The laser light may propagate through free space between the laser and the gas analysis region. The laser light may propagate through free space between the gas analysis region and the detector.

The pulse may be substantially constant over the electrical pulse length and/or wherein the pulse comprises substantially a step function. The electrical pulse may comprise at least one of: a rectangular pulse shape and/or a flat top and/or triangle shaped top

The electrical pulse may minimise the optical output power decay. The method may further comprise selecting one or more parameters of the electrical pulse.

The method may comprise varying the rate of change of wavelength per unit time, for example by varying the amplitude of the current/voltage drive pulse.

The method may comprise adjusting the wavelength scan length, for example, by varying the duration of the current/voltage drive pulse.

The method may comprise varying the semiconductor diode laser temperature.

The method may further comprise comprising adjusting one or more of pulse width, duty cycle, temperature and voltage/current of the pulse.

The method may comprise controlling the starting temperature of the laser with a thermoelectric controller.

The starting temperature may be in a range between −20° C. and 50° C. The starting temperature may be controlled to control the initial wavelength.

The size of the current pulse may be dependent on the laser. The current pulse may have a current in the range between 0.4 A and 1.5 A. The current pulse may be below 2.5 A.

The wavelength scan may comprise size of 0.1 to 10 cm⁻¹.

The wavelength scan may comprise light having a wavelength characterised by wavenumber range. The wavenumber range may have a size in the region of 0.1 to 10 cm⁻¹.

The method may further comprise varying the temperature of the laser.

The output radiation may have a wavelength in the range 0.5 μm to 14 μm.

The semiconductor diode laser may be configured to emit laser light in a near to mid infrared range, optionally in a visible to near range, optionally in a near to mid infrared range.

The produced wavelength chirp may have a size in the range 0.1 to 10 cm⁻¹. The produced wavelength chirp may have a rate of change in the range 0.1 cm 1 to 10 cm⁻¹ per microsecond. The detector and/or digitisation circuitry may be configured to sample emitted light when the wavelength chirp has a rate of change in the range 0.1 to 2 cm⁻¹ per microsecond and/or when the wavelength chirp has a size in the range 0.1 to 5 cm⁻¹.

The optical cell may comprise a Herriott cell. The laser may comprise a semiconductor laser. The laser may comprise a quantum cascade laser, interband cascade laser, or a tunable diode laser.

The method may comprise selecting and/or adjusting one or more operational parameters of the laser to regulate and/or substantially maintain a duty cycle of the laser, for example, to maintain the duty cycle below a target value. The target value may be below 10%, optionally between 2.5% and 7.5%, further optionally below 5%, further optionally between 1 and 5%.

The method may comprise controlling the laser to reduce a pulse repetition rate of the laser, optionally wherein the pulse repetition rate is in the range 0.01 to 5 KHz.

The laser output may be provided to the sample region without further modulation by a further laser.

The wavelength chirp may be caused at least in part, optionally substantially, by a temperature change of the laser caused by the application of the pulse. The change in wavelength is caused by temperature change and is not caused by modulation of the laser, for example, by a second external laser. The produced laser light may comprise unmodulated laser light. The method may comprise wavelength and/or wavenumber tuning due to temperature induced pulse chirping.

In accordance with a second aspect, there is provided a semiconductor diode laser spectrometer for measuring radiation absorption by a sample comprising: a semiconductor diode laser; an electric pulse generator configured to apply an electrical pulse to the laser thereby to cause the laser to produce a laser output pulse for a gas sample region, wherein the application of the electrical pulse to the semiconductor diode laser causes a change in temperature such that the produced laser output pulse comprises a continuous wavelength chirp over a wavelength range wherein the produced laser output pulse is provided to a gas sample region and wherein at least part of the wavelength range of the wavelength chirp is used as a wavelength scan and a detector for detecting optical output from the gas sample region wherein the electrical current pulse comprises an electrical pulse length of at least 1 microsecond. The electrical current pulse may comprise an electrical pulse length of at least 5 microseconds.

The wavelength chirp may be caused, at least in part, by the increase in temperature of the laser induced by the applied electrical pulse The increase in temperature may slow over the length of the current pulse thereby causing the rate of change of the continuous wavelength chirp to continuously slow over the length of the pulse from a first initial rate to a second, slower rate, and wherein the detection and/or further sampling of the optical output is performed at the second, slower rate. The increase in temperature may be due to an increase temperature of the gain or active medium of the laser.

The spectrometer may further comprise an optical cell and wherein at least part of the sample region is in the optical cell. The optical cell may comprise a non-resonant optical cell,

The spectrometer may form part of an open path sensing system. The spectrometer may form part of a cross stack system.

The detector may comprise a photodetector and/or digitising circuitry. The bandwidth of the photodetector may be in the range 1 to 10 MHZ, The bandwidth may be higher or lower depending on the application. The bandwidth of the digitising circuitry may be in the range 1 to 10 MHz. The bandwidth may be higher or lower depending on an application. The detector may comprise detection and digitising circuitry having a bandwidth in the range 1 to 20 MHz.

The detection and digitising circuitry may comprise a photodetector having a bandwidth in the range 1 to 10 MHz and digitizing circuitry having a bandwidth in the range 1 to 10 MHz. The detection and digitising circuitry may comprise a photodetector and digitizing circuitry having a bandwidth in the range 1 to 20 MHz. The detection of the optical input may comprise detection and/or digitising by a photodetector and digitisation circuitry, wherein the detection and/or digitisation is performed when the wavelength chirp is in the range of range of 0.1 to 2 cm⁻¹ per microsecond.

The bandwidth of the digitising circuitry may be greater than the bandwidth of the digitising circuitry. The sampling rate of the digitising circuitry may be at least ten times, optionally at least four times greater than the photodetector bandwidth.

The laser may comprise an intra-pulse laser spectroscopy system, optionally wherein the intra-pulse spectroscopy is configured to detect compounds using direct absorption.

According to a third aspect, there is provided method for sensing gases using a semiconductor diode laser, the method comprising: applying an electrical current pulse to a semiconductor diode laser thereby to cause the laser to produce a laser output pulse, wherein the application of the electrical pulse to the semiconductor diode laser causes an increase in temperature such that the produced laser output pulse comprises a continuous wavelength chirp over a wavelength range; providing the produced laser output pulse to the gas sample region, wherein at least part of the wavelength range of the wavelength chirp is used as a wavelength scan; and detecting optical output from the gas sample region, wherein the wavelength chirp is caused, at least in part, by the increase in temperature of the laser induced by the applied electrical pulse, wherein the electrical current pulse comprises an electrical pulse length, or duration, of at least 5 microseconds and/or wherein the length of the electrical current pulse causes the increase in temperature to slow over the length of the current pulse thereby causing the rate of change of the continuous wavelength chirp to continuously slow over the length of the pulse from a first initial rate to a second, slower rate, and wherein the detection and/or further sampling of the optical output is performed at the second, slower rate.

According to a fourth aspect there is provided a semiconductor diode laser spectrometer for measuring radiation absorption by a sample comprising: a semiconductor diode laser; an electric pulse generator configured to apply an electrical pulse to the laser thereby to cause the laser to produce a laser output pulse for a gas sample region, wherein the application of the electrical pulse to the semiconductor diode laser causes a change in temperature such that the produced laser output pulse comprises a continuous wavelength chirp over a wavelength range wherein the produced laser output pulse is provided to a gas sample region and wherein at least part of the wavelength range of the wavelength chirp is used as a wavelength scan, wherein the wavelength chirp is caused, at least in part, by the increase in temperature of the laser induced by the applied electrical pulse, and a detector for detecting optical output from the gas sample region,

wherein the electrical current pulse comprises an electrical pulse length of at least 5 microseconds and/or wherein the increase in temperature slows over the length of the current pulse thereby causing the rate of change of the continuous wavelength chirp to continuously slow over the length of the pulse from a first initial rate to a second, slower rate, and wherein the detection and/or further sampling of the optical output is performed at the second, slower rate.

Features of one aspect may be provided as features of any other aspects. For example, features of the method may be provided as features of the spectrometer and vice versa. In addition, features of the first aspect may be provided as features of the third aspect and vice versa. In addition, features of the second aspect may be provided as features of the fourth aspect and vice versa.

### BRIEF DESCRIPTION OF DRAWINGS

Various aspects of the invention will now be described by way of example only, and with reference to the accompanying drawings, of which:

FIG. 1 shows a spectrometer arrangement 10 for measuring radiation absorbed by a species, for example, by a gas sample;

FIG. 2(a) is a plot of a laser pulse having a pulse length over 100 microseconds and FIG. 2(b) is a plot of a laser pulse have a pulse length over 10 microseconds;

FIG. 3 shows three pulses ramped with three different rise time constants;

FIG. 4(a) is a plot of an etalon pulse showing tuning and FIG. 4(b) shows a relationship between time and wavenumber for a portion of the etalon pulse of FIG. 4(a);

FIG. 5 is a plot of a voltage, current and light power curve;

FIG. 6(a) is a plot of a pulse against time and FIG. 6(b) is a plot of a pulse against wavenumber, and

FIG. 7(a) is a plot of a current pulse having a flat top and FIG. 7(b) is a plot of a current pulse having a shaped top.

### SPECIFIC DESCRIPTION

WO 03/087787 describes using a pulse generator to provide a plurality of pulses (rectangular) to an input of a semiconductor diode laser. The generator provides a train of fixed amplitude sub-microsecond duration rectangular current drive pulses to cause a fast laser heating effect and hence a continuous wavelength up-chirp of the emitted semiconductor diode laser radiation at a rate in time. The laser heating caused by the current pulses is such that for each pulse emitted from the laser, the chirp is a continuous almost linear spectral variation from short to long wavelength (defined as a continuous spectral or wavelength scan). WO 03/087787 describes applying pulses in the range 150 to 300 ns, preferably 200 to 300 ns to provide a tuning range of about 60 GHz.

In the context of gas analysers, it was previously considered to be of little benefit to pulse lasers such as a semiconductor diode laser beyond around 1000 ns. This view was partly due to the design of the drive circuit which was used. A further factor is the behaviour of the lasers available at that time, for example, the data from WO 03/087787 shows that the characteristics of the lasers at the time were quite different to the lasers that are currently used. Furthermore, in WO 03/087787, the chirp is shown as linear.

Furthermore, in the context of quantum cascade lasers, it was understood that the temperature of a laser may become prohibitively high if pulsed with such long pulses or using a high duty cycle, due to the construction and design of the lasers at the time. It will be understood that the efficiency of the laser decreases as the temperature increases therefore decreasing the light emitted by the laser. However, as described in the following, it has been found that if the laser pulse is sufficiently long then it is possible to produce a useable spectroscopic window. While this requires a larger temporal window to be sampled, the sampling rate may be reduced substantially as the tuning rate in the window of interest is much slower. This results in the ability to use electronic circuitry of lower bandwidth and sampling rate leading to lower cost components. In particular, this reduces the burden on the electronics required in the system, in particular, on the detection side.

The useable spectroscopic window may correspond to a wavenumber range that covers typical absorption lines of desired target gases. Examples of such, without limitation, are provided in Table 1. Without limitation, the range may typically be around 0.05 to 0.1 cm 1, therefore requiring a minimum total chirp having a size of 0.1 to 0.2 cm⁻¹. For more complex spectroscopic cases a larger wavenumber range may be required. The useable spectroscopic window will therefore be understood as a window that allows accurate identification and measurement of one or more gases of interest.

It will be understood that the produced wavelength chirp may have a duration, measured in microseconds. The produced wavelength chirp may also be characterised by a size in terms of wavenumbers. In some embodiments, the size of the produced wavelength chirp is in the range 0.1 to 10 cm⁻¹. In some embodiments, the rate of change of the wavelength chirp is in the range 0.1 to 10 cm 1 per microsecond.

As described above, only a portion of the chirp provides useable data. In some embodiments, this portion may correspond to a proportion of the wavelength chirp, for example, a quarter, half or three quarters of the wavelength chirp. In some embodiments, the useable portion corresponds to part of the wavelength chirp that has a rate of change below a threshold value or in a suitable or desired range. In some embodiments, the useable portion corresponds to part of the wavelength chirp with a rate of change in the range 0.1 to 2 cm⁻¹ per microsecond. In some embodiments, the useable portion has a size in the range 0.1 to 5 cm⁻¹. The useable portion of the chirp may correspond to the useable spectroscopic window. The useable portion may have a duration that defines or corresponds to a detection window.

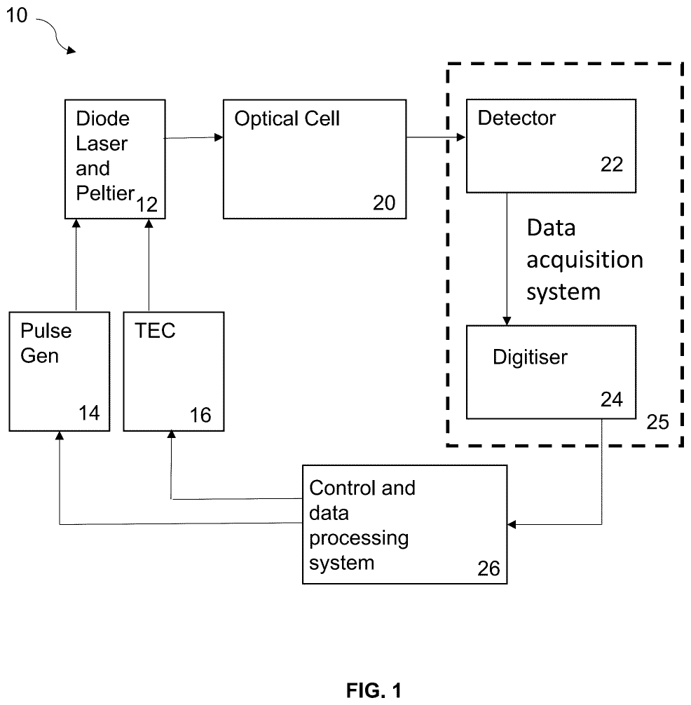

FIG. 1 shows a spectrometer arrangement 10 for measuring radiation absorbed by a species, for example, by a gas sample. The spectrometer 10 has a semiconductor diode laser 12 and a current drive pulse generator 14 connected to input of the laser 12. The pulse generator 14 is operable to provide substantially rectangular pulses to the laser 12. In the present embodiment, the laser 12 is a single mode semiconductor diode quantum cascade laser (QC laser). The laser 12 is mounted on a Peltier temperature (not shown in FIG. 1) and enclosed within a laser package (or housing) that is adequately heatsinked. The Peltier element is controlled by a thermoelectric controller 16 (TEC). In the present embodiment, the output radiation has a wavelength in the range 0.5 μm to 14 μm. The Peltier element is controlled by a thermoelectric controller 16 which allows the starting temperature of the laser to be configured. This allows the starting wavelength of the laser to be selected.

The spectrometer of FIG. 1 has a closed non-resonant optical cell 20 for collecting a sample of gas to be analysed. According to embodiments, the gas being analysed may be contained within a Herriot cell, an astigmatic Herriot cell, other types of multi-pass cell, or an open-path arrangement may be used.

In the arrangement of FIG. 1, radiation emitted by the semiconductor laser traverses an optical path through the optical cell 20. The light output from the optical cell 20 is directed (by steering optics if necessary) to a photodetector 22, also referred to as a detector. The detector 22 detects the absorbed light pulse output from the optical cell 20. Connected to the detector 22 is a digitiser 24, which is also connected to a control and data processing system 26. The detector and digitiser form a data acquisition system 25. The control and data processing system 26 provides overall control of the spectrometer. In addition to the digitiser, the control and data processing system 26 is connected to the current drive pulse generators 14.

In some embodiments, more than one laser is provided. In such embodiments, each laser may have their own pulse generator and TEC. In some embodiments, more than one detector and digitiser is provided.

As part of its functionality, the control system 26 is operable to set the amplitude and duration of the pulses applied to the laser and monitor the resultant outputs detected from the optical cell 20.

In the present embodiments, the control system 26 is operable to determine the ratio Iₐ/Iₒ. This could be done using, for example, Beer-Lambert's Law, which may be written as Iₐ/Iₒ=exp (−aL). Of course, as will be appreciated by the skilled person, other techniques could be used. In the low intensity limit, the spectrometer determines the absorption coefficient of a species by measuring the ratio of the intensity of the light incident on the sample gas cell, Iₒ and that transmitted through a sample gas cell containing the absorbing species, Iₐ. In the low intensity limit, the change in the intensity of light that passes through the gas is described by the Beer-Lambert relationship, Iₐ=Iₒ exp (−aL), with the absorption coefficient and L the optical path length. It should be noted that a is a function of wavenumber and is independent of the intensity at low intensities of the incident radiation.

It will be understood that the controller may be configured to sense a gas or sense one or more compounds in a sample of gas, or detect one or more materials in a sample of gas. Non-limiting examples are provided with reference to Table 1. Furthermore, the controller may be configured to identify one or more compounds in a sample of gas. The photodetector 22 and digitiser 24 may form or be referred together as a detection system or module.

In the present embodiment, the photodetector 22 is configured to produce an electrical signal in response to laser light incident on the detector 22. The digitiser 24 is connected to the detector 22 and is configured to perform a sampling or digitisation process on the electrical signal of the detector 22. The sampling and digitisation system generates data to be processed by a processing resource. The processing resource may be provided as part of the controller or as an additional processing resource.

Turning to the pulse generator 14, the pulse generator 14 drives the laser 12 to produce laser light. An up-chirp (also referred to as an “effective emission linewidth”) is induced by the temporal duration of the drive current pulse applied to the laser. The term “effective emission linewidth” here means the observable/measurable spectral width (FWHM) of the emission of a semiconductor diode laser induced by an applied current/voltage pulse to its electrical contact. For example a 10 us pulse would have a 6 wavenumber chirp which corresponds to a frequency variation of about 60 GHz. As a further example, a 1 us pulse has typically 2 wavenumber chirp corresponding to a frequency variation of about 20 GHz.

In the present embodiment, the pulse generator 14 is configured to generate a plurality of substantially rectangular pulses that are applied to the input of the laser 12. More specifically, the generator 14 provides a train of fixed amplitude rectangular current drive pulses. Each pulse has an electrical pulse length of at least 1 microsecond (1000 ns). This causes a laser heating effect and hence a continuous wavelength up-chirp of the emitted semiconductor diode laser radiation. In embodiments, the electrical pulse length has a duration of at least 5 microseconds (5000 ns). The pulse length has a duration that is long enough that the chirp rate of the produced chirp slows to a slower rate.

The pulse is substantially constant over the electrical pulse length. Each pulse may also be considered to be represented mathematically as a step function or a suitable approximation. The pulse may be considered to be a rectangular pulse shape.

The laser heating caused by the rectangular current pulses is such that for each pulse emitted from the laser, the chirp is a continuous spectral variation from short to long wavelength. This may be considered to define a continuous spectral or wavelength scan. A chirp is understood as a change of wavelength over time. The chirp may be characterised by a chirp rate. The chirp rate is a change in chirp over time. In WO 03/087787, the chirp is linear and therefore the chirp rate is constant and does not change over time. In contrast, as described in the following, the chirp is non-linear and the chirp rate slows down during the pulse.

It will be understood that one or more properties of the wavelength chirp are dependent on the temperature of the laser. The temperature of the laser changes as laser light is produced, in particular, the temperature of the laser increases as the electrical pulse is applied up to a maximum temperature. The maximum temperature possible will be dependent on the laser characteristics and environmental factors, such as the ambient temperature and/or any cooling effects, for example, from the TEC. The application of the electrical current pulse to the semiconductor thus provides a heating effect to the semiconductor laser.

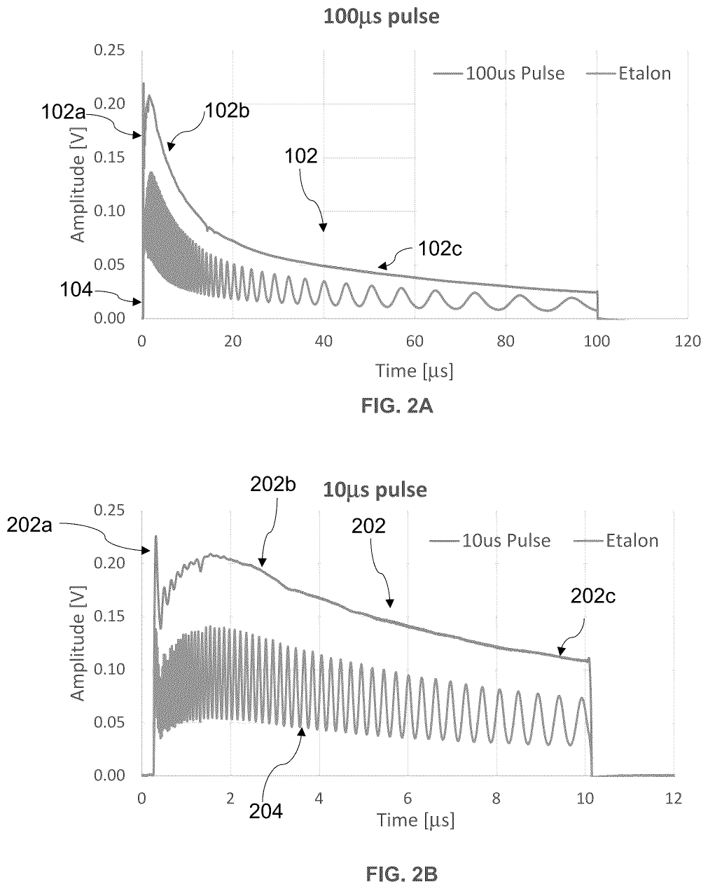

It has been found that a pulse length over 1 microsecond is sufficiently long to allow rate of temperature increase of the laser to slow over the application of the pulse. In embodiments, this effect may be enhanced by using a chirp duration longer than 1 microsecond, for example, greater than 5 microsecond. In further embodiments, the chirp duration may be up to and greater than 1 millisecond. As such, the rate of change of the wavelength of the produced chirp slows over time. This effect is demonstrated, for example, in FIGS. 2, 3 and 4 below. The chirp rate thus decreases over the wavelength chirp. This offers advantages, for example, in that the portion of the sampled photodetector signal analysed has a wavelength chirp rate that allows optimisation of the detector and acquisition system. As a further example, the laser may be configured to scan the wavelengths of interest at a rate where the signal has lower electrical bandwidth allowing the use of a lower cost photodetector and acquisition system. As a further example, it may also allow the spectral resolution to be higher because the chirp rate is slower.

In operation a response to the entire pulse signal is detected by the photodetector to produce a photodetector signal and the entire photodetector signal is sampled and/or digitised. However, due to the bandwidth of the photodetector and the digitising circuitry, only part of the sampled signal is useful.

The system can target a desired spectral window by adjusting laser parameters. The system can be configured so that the useful part of the spectral window corresponds to one or more spectroscopic features of interest, for example one or more absorption lines. The system can be configured so that the spectroscopic features of interest occur during the slower part of the pulse.

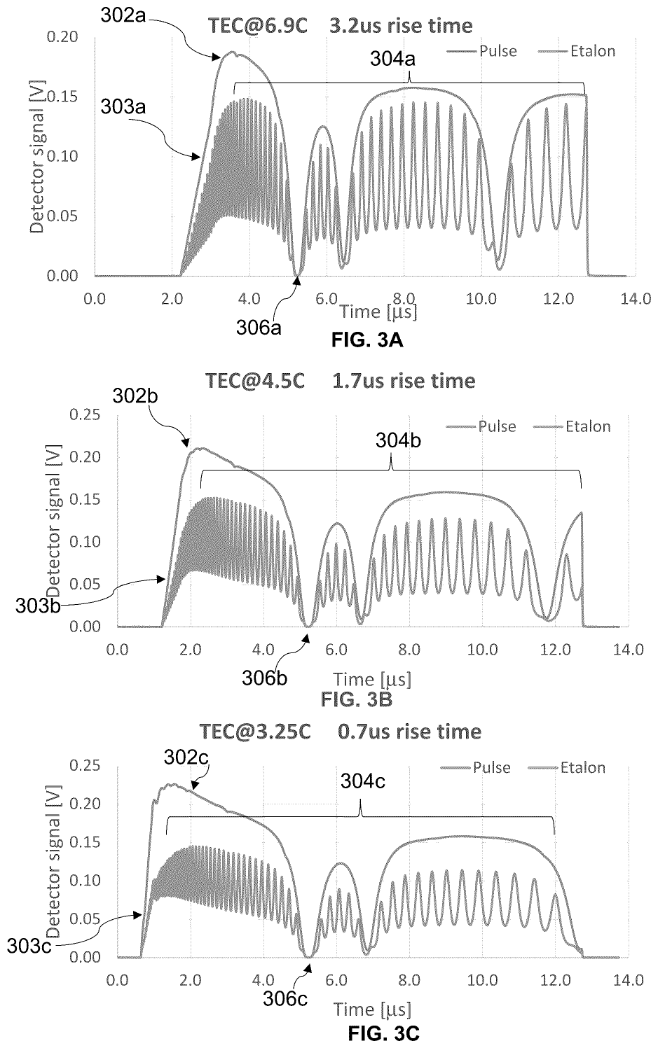

In some embodiments, the wavelength chirp comprises a portion that corresponds to a chirp rate in a desired range. That portion occurs during a time period that is referred to as a detection time window. The controller may be configured so that the sampling and detection is performed during at least the desired detection time window to allow detection data from the desired window to be acquired. As described in further detail, for example, with reference to FIG. 3, the TEC can control the temperature to define a start of the chirp. In some embodiments, a synchronization process is performed to ensure detection is performed during at least the desired detection time window.

It will be understood that data is captured but only some is selected for processing. As described above, all detection data is captured by the detection and sampling process to provide a reference value, as it is a relative amplitude that is being measured. The detector may have an off-set that is non zero.

The slower chirp rate reduces the frequency content of the electrical signal from the photodetector therefore reducing the bandwidth required for both the photodetector and electronics used to digitise the signal. A lower bandwidth may result in a number of advantages. As described above, the chirp in a long laser pulse is fast at the start of the pulse, but slows down later in the pulse. By calibrating the laser to sweep the wavelength of interest when the chirp is slower, the electrical frequency content of the pulse signal produced is reduced.

In the present embodiment, one or more operational parameters of the pulse generator or the laser are selected in order to regulate and/or substantially maintain a duty cycle of the laser. In particular, the laser is operated at a low duty cycle to maintain a low average heat load. In the present embodiment, the pulse repetition rate is controlled to be in a desired range. In the present embodiment, the pulse repetition rate is maintained in the desired range 0.01 to 5 KHz. This allows the duty cycle to be maintained below a desired level or predetermined value. In the present embodiment the desired level is a target value of 5%. In further embodiments, the target value may be a value below 10% or, for example, a value between 2.5% to 7.5%. In addition, the temperature is controlled by the TEC to define the start of the chirp and/or the position of the desired spectroscopic or detection time window. As described above, the TEC defines the start of the chirp and the laser reaches an equilibrium for certain operating conditions.

In addition to advantages relating to reducing the burden on electronics, the wavelength chirp itself is relatively longer than those produce using a shorter pulse. This allows a larger range of wavelengths or larger range of wavenumbers to be measured from a single laser.

In operation, an electrical pulse is applied to the semiconductor diode laser to cause the laser to produce a laser output pulse. The electrical pulse has a pulse length of at least one microsecond. The applied electrical pulse causes a change in temperature of the laser such that the produced laser output pulse has a continuous wavelength chirp over a wavelength range. The produced output pulse is then provided to a gas sample region, in this embodiment, in an optical cell 20 and interacts with a sample of gas.

Following interaction with the gas, light that has passed through the optical cell 20 is incident on the photodetector 22. The detector 22 is configured to produce a detection signal in response to the light being incident on the detector 22. In the present embodiment, the control system 26 is configured to synchronise operation of the pulse generator 14 and the digitiser 24 such that the digitiser performs a sampling process on the detection signal. In the present embodiment, a sampling process is performed over the wavelength chirp allowing detection data to be captured during a time period in which the wavelength chirp is changing at a slower rate. The detection data from this part is then processed to determine, for example, the presence and/or absence of one or more compounds of interest in the sample of gas.

It will be understood that a number of parameters of the electrical pulse may be selected. For example, the pulse length may be varied. As described above, the electrical pulse length is over 1 microsecond. In some embodiments, the electrical pulse length is between 1 microsecond and 200 microseconds. In further embodiments, the electrical pulse length is between 100 microseconds and 10 milliseconds. In other embodiments, the electrical pulse length is between 100 microseconds and 1 millisecond. A variation in the electrical pulse length allows the wavelength scan length to be adjusted. Other parameters of the pulses may also be adjusted by the control system. For example, one or more of pulse width, duty cycle, temperature and current of the pulse may be controlled.

In embodiments, the electrical pulse length is between 5 microseconds and 1 millisecond. In embodiments, the electrical pulse length is between 10 microseconds and 1 millisecond.

In addition, parameters of the pulse or other parameters of the system may be varied in order to vary the rate of change of wavelength over time. For example, the amplitude of the current pulse may be varied to provide for a change in the rate of change of wavelength per unit time.

In comparison to known systems in which the laser pulse frequency is in the range 1 to 50 kHz, the laser pulse frequency may be in the region 10 Hz to 5 kHz. In comparison to known systems in which a typical value of the bandwidth of the photodetector was in the range 80 to 100 MHZ it has been found that the bandwidth of the photodetector may be an order of magnitude lower, for example, this may be in range 1 to 10 MHz.

In some embodiments, the laser pulse frequency is between 100 Hz and, for example, 40 KHz. As a non-limiting example, the pulse width may be 10 microseconds which would be a 5% duty cycle when operating at 5 kHz.

It will be understood that a lower bandwidth photodetector and/or digitiser may be used because the bandwidth requirement is reduced. The bandwidth required is reduced because the chirp rate of the spectroscopic portion of interest is slower because the pulse is longer.

In some embodiments, the electrical pulse is such that the optical output power decay is minimised. It will be understood that the laser drive system is optimized for driving pulses in the pulse length between 5-100 μs. In further detail, any drop in the laser drive current, exhibited by some lasers drives (for example, those used for sub microsecond pulsing) is minimized to avoid an excessive decay in the optical output of the pulse.

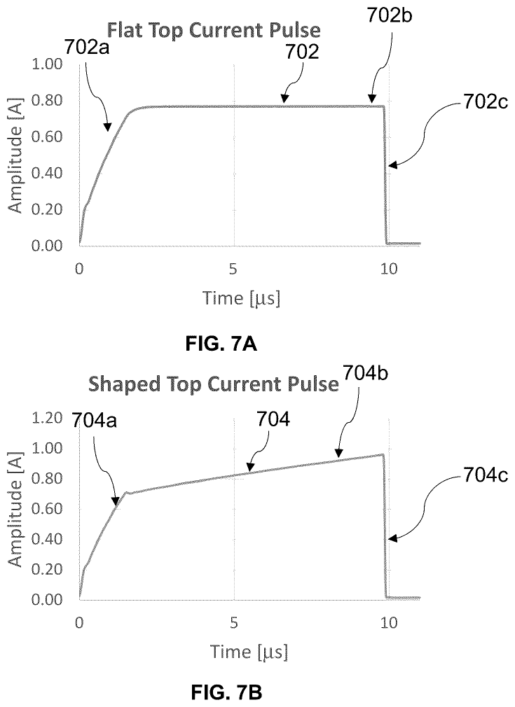

In the above described embodiments, a rectangular current pulse shape was described. In further embodiments, the shape of the electrical current pulse may be adjusted to improve the usefulness of the optical signal produced by the laser. This includes, but is not limited to, controlling a rise time of the current pulse to optimize both the optical power output of the laser and the wavelength turning during the first part of the pulses, where the chirp rate is faster than the photodetector and data acquisition system is configured to properly capture. In some embodiments, the electrical current pulse may have a flat top (i.e. a period of constant current), or may be shaped (such that the current changes during the middle part of the pulse). The shape of the pulse may be selected to adjust the wavelength chirp that the laser exhibits to achieve the optimum behaviour for a specific laser or measurement. FIG. 7 depicts current pulses having different shapes.

FIG. 2(a) shows a detector signal resulting from a long laser pulse. The pulse length in FIG. 2(a) is 100 μs. The upper trace 102 is a detector signal of the quantum cascade laser. The upper trace may be considered as a temporal pulse or temporal response. The lower trace 104 is an etalon trace depicting the fringes observed after the pulse has passed through an etalon. In the present embodiment, the etalon is a Germanium etalon having a length of half inch. The frequency of the etalon trace decreases over time demonstrating a non-linear spectral variation from short to long wavelength over the pulse.

As can be seen in FIG. 2(a), the pulse has a first portion 102a in which the signal amplitude increases rapidly. The first portion may also be referred to as a fast turn on portion. The pulse has a second portion 102b and a final portion 102c. The corresponding portions of the etalon trace 104 show a non-linear spectral variation from short to long wavelength over the duration of the pulse. In particular, the chirp rate slows down during the pulse.

FIG. 2(b) shows a detector signal from a long pulse with pulse length 10-μs. The upper trace 202 is the detector signal and the lower trace 204 is an etalon trace showing an etalon fringe pattern. The detector signal has a first portion 202a, a second portion 202b and a third portion 202c. Between the second portion 202b and the third portion 202c is a local minimum corresponding to absorption. The corresponding portions of the etalon trace 104 show a non-linear spectral variation from short to long wavelength over the duration of the pulse. In particular, the chirp rate slows down during the pulse.

It will be understood that the plots of FIGS. 2(a) and 2(b) represent averaging of the signal responses over 100 to 1000 pulses per second. These were then recorded with an oscilloscope, averaging 32 consecutive such pulse trains.

FIG. 3 illustrates the effect of applying a ramp of different rates to the pulse current. In contrast to known systems in which the current and pulses are turned on suddenly and controlled by square voltage pules, FIG. 3 shows a ramp applied to the pulse current.

FIG. 3 shows three pulses ramped with three different rise time constants. The upper trace in each of FIGS. 3(a), 3(b) and 3(c) (labelled 302a, 302b and 302c) are plots of the pulse against time and the lower trace is the etalon trace. The rise time constants are applied to the current by configuring the pulse driver.

FIG. 3(a) shows a result of a first pulse 302a ramped at a first rate (with a 3.2 μs rise time). FIG. 3(b) shows a result of a second pulse 302b ramped at a second rate (with a 1.7 μs rise time). FIG. 3(c) shows a result of a third pulse 302c pulse ramped at a third rate (with a 0.7 μs rise time). Each plot has a first rising portion (303a, 303b, 303c) followed by a second descending portion (304a, 304b, 304d)

For the three pulses, different TEC temperatures are used to control a timing of a reference line (in this example, the first reference line from the left). In this example, the reference line is maintained at 7.5 μs from the end of the pulse. The reference line is labelled as 306a, 306b and 306c in each Figure respectively.

FIG. 3 demonstrates that faster rise times (for example, FIG. 3(a)) result in a smaller useable spectral window from the reference line to the end of the pulse. However for the same temperature a faster ramp will lead to more overall useable tuning. Therefore, as a compromise to achieve both long tuning and to maximise the portion of in the second, more slowly tuning part of the pulse, an intermediate rise time is selected. It will be understood that the value for the intermediate rise time may be dependent on the laser. There may be advantages to both faster and slower rise times, so it is about optimising for the value that is best for the overall system. 1.7 μs is an arbitrary number that has been show to work effectively. As a non-limiting example, the value 1.7 μs was used. However, it will be understood that a different value may be used, for example, a value in the range 1 to 2 μs may be used.

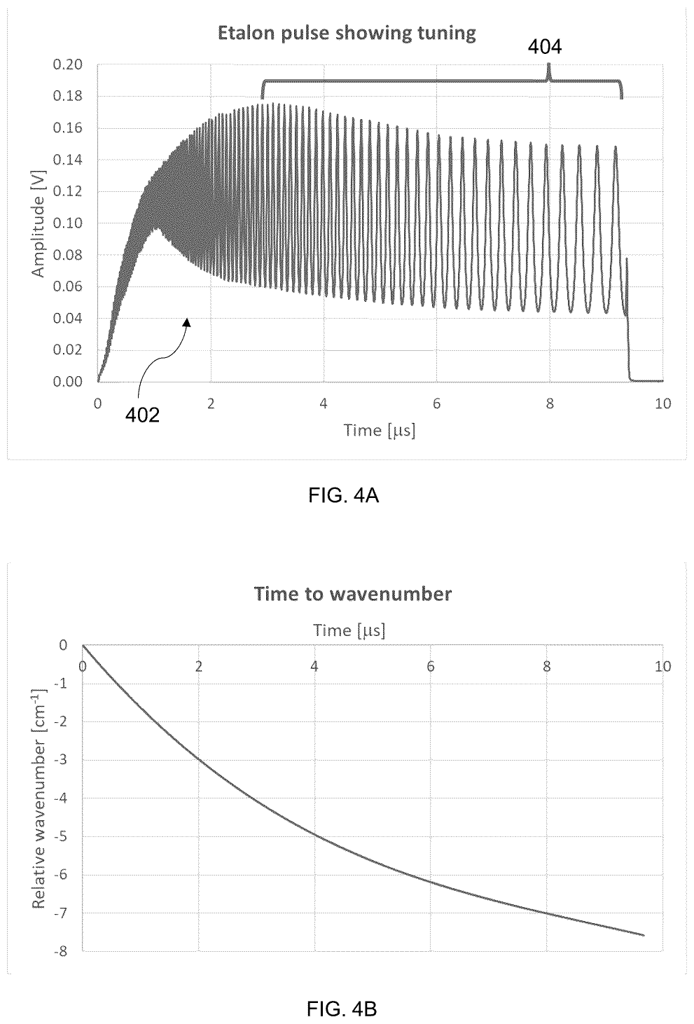

FIG. 4(a) depicts an etalon pulse trace 402 that shows tuning. The y-axis is the photodiode signal and the x-axis is the time. A portion 404 of the pulse trace 402 is indicated. The portion 404 may be referred to as the useable portion and may correspond to a useable spectral window. The useable spectral window refers to the portion of the pulse trace that is typically better suited for spectroscopy. In some embodiments, data collected outside the useable portion 404 is discarded and not used for data processing.

FIG. 4(b) depicts the relative time to wavenumber conversion for the entire pulse. The y-axis is the relative wavenumber in units of cm\hat{ }−1. The x-axis is the time in units of μs. The chirp rate, corresponding to the change in wavenumber over time, slows continuously over the duration of the pulse. The chirp is non-linear over the duration of the pulse. The chirp rate therefore decreases significantly over the length of the pulse. The chirp rate decreases continuously over the length of the pulse. The chirp rate can be considered to be a monotonically decreasing.

The non-linearity of the chirp may be due to a number of factors. Without being bound by theory, these may be due to effects such as thermal heating effects of the laser, external cooling effects and changes in a refractive index. As such, it can be complex to precisely model the non-linearity of the chirp. For the purposes of obtaining useable output from the detector an approximation can be used. This may be a polynomial function or exponential function. In some embodiments, a polynomial of at least third order may be need to represent the non-linearity due to the longer pulse length. A multi-term exponential function may be used to represent the chirp.

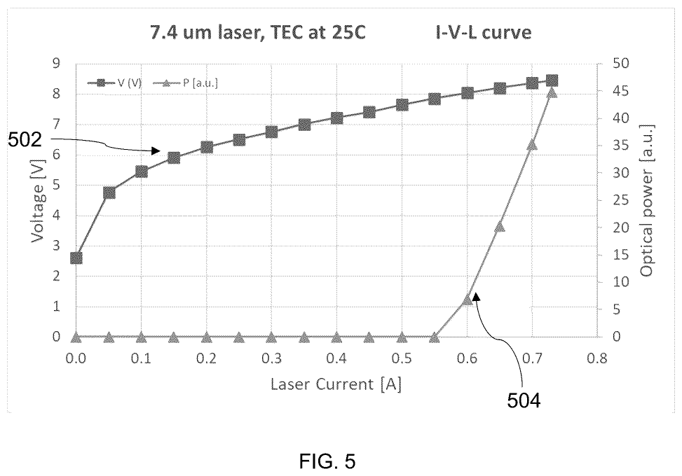

FIG. 5 depicts an I-V-L curve. A first plot 502 shows variation in voltage (in volts) against laser current (in amps). A second plot 504 shows variation in light power against laser current. The limiting values of light power are defined, at the lower end, by the current/voltage amplitude necessary to achieve a usable output power and at the upper end, by the current/voltage amplitude that induces a reduction in the output power. The upper limit is based on the manufacturer's maximum specification and may be where the output power reduces or where other characteristics of the laser output can no longer be maintained. The lower limit is defined by an optical threshold of the laser, i.e. the minimum current required for an emission of laser light.

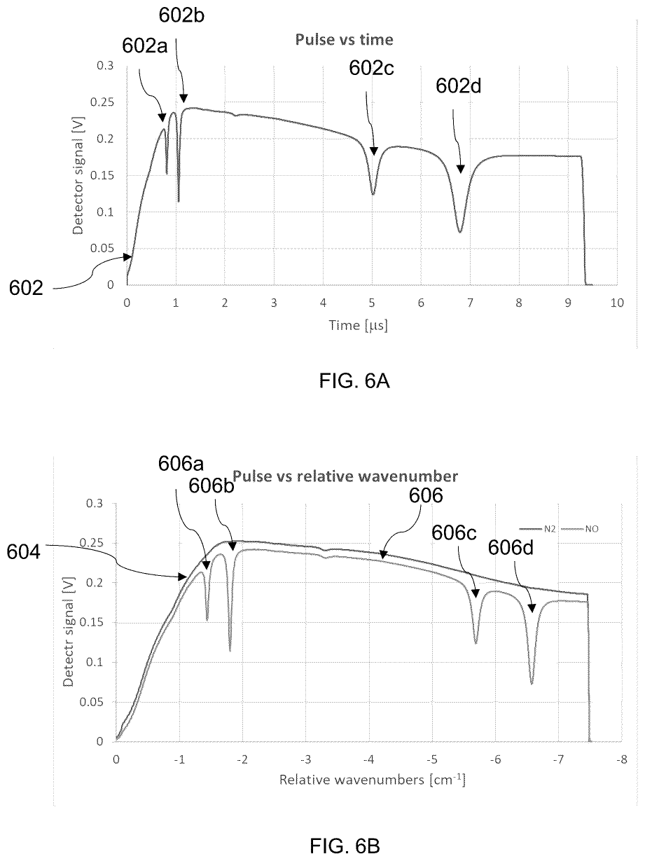

FIG. 6(a) depicts a plot of the photodiode signal versus time. Curve or signal 602 corresponds to the detector signal obtained with the measurement cell filled with target gas (in this example NO). The curve 602 has a number of signal features (602a, 602b, 602c, 602c) corresponding to absorption of the laser light by the target gas. The signal features correspond to absorption lines of the target gas. Signal features may also be referred to as signatures.

FIG. 6(b) depicts the photodiode signal of the pulse against relative wavenumber. Both plots were acquired using a 10 μs pulse for a 5 μm laser. Curve 604 corresponds to a signal obtained when the measurement cell is filled with N₂. Curve 606 corresponds to a signal obtained when the measurement cell is filled with the target gas (NO) in N₂ balance. Curve 606 thus exhibits a number of signal features (606a, 606b, 606c, 606d) correspond to absorption lines of the target gas.

Comparing FIGS. 6(a) and 6(b), the signal features of the signal obtained over time (represented by 602a, 602b, 602c, 602d) correspond to the signal features of the signal obtained against wavenumbers (606a, 606b, 606c, 606d). Such a correspondence will be understood due to a continuous wavelength chirp produced by the laser that is provided to the measurement cell. The produced laser output provided to the measurement cell has a changing wavenumber (over time) that provide a wavelength or wavenumber scan.

FIG. 7(a) depicts a current pulse having a flat top. The flat top current pulse 702 has a first portion 702a, a second portion 702b, and a third portion 702c. The first portion 702a is increasing in amplitude over time. The flat top portion has a portion that has a constant current value over a period of time (corresponding to second portion 702b).

FIG. 7(b) depicts a current pulse 704 having a shaped top. The current pulse has a first portion 704a, a second portion 704b, and a third portion 704c. In contrast to the current pulse of FIG. 8(a), the second portion 704b is not flat and not constant in time, but increasing in time. The first portion 704a increases at a first rate, and the second portion 704b increases at a second, larger rate.

Table 1 presents example values of wavelength range and corresponding wavenumber range for sets of compounds. It will be understood that these examples are provided as non-limiting and alternative compounds and/or groups of compounds may be detected using the method and system described above.

TABLE 1

| | wavelength | Wavenumber | |
|---|---|---|---|
| Laser | range (μm) | range (cm⁻¹) | Compounds detected |
| 1 | 5.2356-5.2632 | 1900-1910 | Nitric oxide (NO), Water (H2O) |
| 2 | 6.1162-6.1538 | 1625-1635 | Nitrogen dioxide (NO2), Water |
| | | | (H2O), Ammonia (NH3) |
| 3 | 4.4743-4.4944 | 2225-2235 | Carbon Monoxide (CO), |
| | | | Carbon Dioxide (CO2) |
| 4 | 7.4349-7.4627 | 1340-1345 | Methane (CH4), Sulfur Dioxide |
| | | | (SO2) |
| 5 | 10-10.2 | 980-1000 | Ammonia (NH3), Acetylene |
| | | | (C2H2) |
| 6 | 4.7281-4.7393 | 2110-2115 | Carbon Monoxide (CO) |
| 7 | 4.8544-4.8780 | 2050-2060 | Carbon Monoxide (CO), |
| | | | Carbon Dioxide (CO2) |
| 8 | 4.2194-4.2373 | 2360-2370 | Carbon Dioxide (CO2) |

A skilled person will appreciate that variations of the disclosed arrangements are possible without departing from the scope of the invention. For example, in the above described embodiments, the laser is a quantum cascade laser. However, it will be understood that other semiconductor lasers configured to produce wavelength chirps may be used, for example, interband cascade laser, tunable diode laser. Furthermore, in the above embodiment, a single mode laser was described, however, in alternative embodiments, a multimode laser is provided. In such embodiments, an optional spectral filter is provided on the optical path between the diode laser and the optical cell that is controlled by the control and acquisition system. The spectral filter may be, for example, a small grating monochromator, and may be used to provide a single mode laser output if a multi-longitudinal mode laser is used. In some embodiments, additional optical elements may be provided in the setup, for example, between the laser and the cell.

In accordance with embodiments, the wavelength range of the lasers may be near IR to mid IR. Near infrared may comprise the range 800 to 2500 nm. Mid infrared may comprise the range between 2.5-3 micron to about 25 micron. Far infrared may correspond to range beyond 25 micron up to mm. Applications of the present systems and methods for Molecular spectroscopy may fall into the near to mid infrared range. In non-limiting examples, quantum cascade lasers used may have an output wavelength range between 3-10.1 μm. In addition, in some examples, tunable diode lasers and ICLS in the range of 0.76 to 4 μm may be used.

In addition, in the above described embodiment, a system using an optical cell is described. However, it will be understood that the system can be extended to alternative gas sensing systems, for example, an open path sensing system, for example, a cross stack system.

As a further non-limiting example, in the above-described embodiment, a rectangular pulse shape was described. However, it will be understood that other pulse shapes may be used, for example, a flat top and/or triangle shaped top.

With a slow ramp turn on, the intensity of the light during the unusable region of the pulse is reduced therefore allowing optimisation of signal levels. The turn on shape of the pulse may also be controlled to allow us to optimise the chirp rate characteristics of the laser which can be used.

Further, in the above-described embodiments, the gas sample region was described as inside a multi-pass optical cell, however, it will be understood that other configurations may be provided.

The rise in temperature caused by the application of the pulse is dependent on the laser being used. In some embodiments, for a particular laser under particular operating conditions, a change in temperature may corresponds to a chirp size. Approximately and without limitation, 1 degree C. of temperature rise may correspond to a portion of the wavelength chirp having a size 0.1 cm\hat{ }⁻¹.

In an example embodiment, the rise in temperature may be between 50 to 80 degrees. That rise in temperature may correspond to wavelength chirp having a size between 5 cm\hat{ }⁻¹ to 8 cm\hat{ }⁻¹.

While dependent on the laser, for example, on the laser properties including the laser wavelength, in some embodiments, the wavelength chirp may span one wavenumber for every 10 degree C. increase in temperature. In an example embodiment, a chirp may span 3 to 4 wavenumbers (3 to 4 cm⁻¹) corresponding to an approximate a 30 to 40 degree C. increase in temperature.

In accordance with embodiment, as described above, the useable window will be a fraction of the wavenumber spanned by the chirp. Without limitation, the useable window may be 0.5 to 1 wavenumbers (0.5 to 1 cm⁻¹). The useable window may be up to 75%, optionally up to 50%, optionally up to 25%, optionally up to 10% of the size of the total wavelength chirp.

As a comparative example, a pulse length under 1 μs would be typically produce a chirp having a chirp rate between 2 cm⁻¹/μs and 7 cm⁻¹/μs. Such systems require a faster detection and/or digitisation system with a bandwidth around 100 MHz.

By providing a method that allows for use of a lower bandwidth detector, a more cost-effective and technically efficient method and system may be provided.

In accordance with a further aspect, there is provided a method of using a QCL or ICLs in a gas analyser where the pulse is longer than was previously possible with pulsed QCLs. Exploiting the wavelength change due to temperature rise caused by the pulse, which slows down significantly with a longer pulse which enables the use of lower speed and/or lower bandwidth detection systems.

In the above described embodiments, different types of semiconductor diode lasers configured to output different wavelengths of laser light are discussed. In embodiments, the quantum cascade lasers may output mid infrared range laser light. In embodiments, the quantum cascade lasers may output light having a wavelength between 3 and 11 μm. In further embodiments, the system may use tuneable diode lasers and interband cascade lasers. In further embodiments, the system may use tuneable diode lasers and interband cascade lasers. TDLs and ICLs having an output wavelength between 0.7 to 4 μm may be used.

In some embodiments, the laser is configured to output laser light having visible to near infrared wavelength. The laser is configured to output laser light having near to mid infrared wavelength.

The detection system may be an intra-pulse laser spectroscopy system. Intra-pulse in this context may be understood as performing detecting of gases during the pulse duration. The system may also be described as a direct absorption system, in which absorption of the light having a wavelength indicates presence of a compound of interest.

In an intra-pulse system or modulation scheme, the chirp may be resolved with a detector in order to scan through an absorption line/signature. Accordingly, the above description of the specific embodiment is made by way of example only and not for the purposes of limitation. It will be clear to the skilled person that minor modifications may be made without significant changes to the operation described.

1. A method for sensing gases using a semiconductor diode laser, the method comprising:
applying an electrical current pulse to a semiconductor diode laser thereby to cause the laser to produce a laser output pulse, wherein the application of the electrical pulse to the semiconductor diode laser causes an increase in temperature such that the produced laser output pulse comprises a continuous wavelength chirp over a wavelength range;
providing the produced laser output pulse to the gas sample region, wherein at least part of the wavelength range of the wavelength chirp is used as a wavelength scan; and detecting optical output from the gas sample region,
wherein the wavelength chirp is caused, at least in part, by the increase in temperature of the laser induced by the applied electrical pulse,
wherein the electrical current pulse comprises an electrical pulse length, or duration, of at least 5 microseconds and/or
wherein the length of the electrical current pulse causes the increase in temperature to slow over the length of the current pulse thereby causing the rate of change of the continuous wavelength chirp to continuously slow over the length of the pulse from a first initial rate to a second, slower rate, and wherein the detection and/or further sampling of the optical output is performed at the second, slower rate.

2. The method as claimed in claim 1, wherein the current pulse may be sufficiently long that the increase in temperature of the laser slows over the electrical pulse length causing the chirp to be non-linear and/or the rate of change of the produced wavelength chirp to slow over the produced wavelength chirp.

3. The method as claimed in claim 1, wherein at least one of:
a) the chirp may be represented by a polynomial or exponential function or other non-linear function;
b) the current pulse comprises a length or duration such that the continuous wavelength chirp is non-linear in time.

4. The method as claimed in claim 1, wherein the temperature change comprises an increase up to a maximum temperature, wherein the maximum temperature is dependent on one or more of an ambient temperature and a cooling effect.

5. The method as claimed in claim 1, wherein the wavelength chirp comprises a portion characterized by a chirp rate in a desired range, wherein the portion occurs during at least a detection time window wherein the method comprises detecting output from the sensing region and/or processing detection signals during at least the detection time window.

6. The method as claimed in claim 5, wherein at least one of:
a) the detection window start at a time beyond 1 microsecond from the start of the pulse;
b) the start of the detection portion is fixed and/or is synchronized with the laser output.

7. The method as claimed in claim 1, wherein the electrical current pulse may comprises an electrical pulse length optionally at least 5 microsecond, optionally at least 10 microseconds, further optionally between 5 microseconds and 1 millisecond, further optionally between 10 microseconds and 1 millisecond.

8. The method as claimed in claim 1, wherein the first rate is above 2 cm⁻¹ per microsecond and the second rate is below 2 cm⁻¹ per microsecond, optionally wherein the first rate is above 2 cm⁻¹ per microsecond and the second rate is in a range of 0.1 to 2 cm⁻¹ per microsecond, further optionally wherein the second rate is below 0.1 cm⁻¹ per microsecond.

9. The method as claimed in claim 1, wherein the chirp rate may slow by up to 25%, optionally 50%, optionally over 100%.

10. The method as claimed in claim 1, wherein the pulse is substantially constant over the electrical pulse length and/or wherein the pulse comprises substantially a step function.

11. The method as claimed in as claimed in claim 1, wherein the electrical pulse comprises at least one of: a rectangular pulse shape and/or a flat top and/or triangle shaped top.

12. A method as claimed in as claimed in claim 1 further comprising at least one of a), b), c), d):
a) varying the rate of change of wavelength per unit time, for example by varying the amplitude of the current/voltage drive pulse;
b) adjusting the wavelength scan length, for example, by varying the duration of the current/voltage drive pulse;
c) varying the semiconductor diode laser temperature;
d) adjusting one or more of pulse width, duty cycle, temperature and voltage/current of the pulse;
e) controlling the starting temperature of the laser with a thermoelectric controller.

13. The method as claimed in as claimed in claim 1, wherein the wavelength scan comprises a size in the range 0.1 to 10 cm⁻¹.

14. The method as claimed in claim 1, wherein at least one of a), b):
a) the output radiation has a wavelength in the range 0.5 μm to 14 μm;
b) the semiconductor diode laser is configured to emit laser light in a near to mid infrared range, optionally in a visible to near infrared range, optionally in a near to mid infrared range.

15. The method as claimed in claim 1, wherein at least one of a), b), c):
a) the produced wavelength chirp has a size in the range 0.1 to 10 cm⁻¹;
b) the produced wavelength chirp has a rate of change in the range 0.1 cm⁻¹ to 10 cm⁻¹ per microsecond;
c) the detector and/or digitisation circuitry is configured to sample emitted light when the wavelength chirp has a rate of change in the range 0.1 to 2 cm 1 per microsecond and/or when the wavelength chirp has a size in the range 0.1 to 5 cm⁻¹.

16. The method as claimed in claim 1, wherein the gas sample region comprises an optical cell, optionally a Herriott cell.

17. The method as claimed in claim 1, wherein the laser comprises a semiconductor laser, optionally, wherein the laser comprises a quantum cascade laser, inter-band cascade laser, tuneable diode laser.

18. The method as claimed in claim 1, further comprises selecting and/or adjusting one or more operational parameters of the laser to regulate and/or substantially maintain a duty cycle of the laser.

19. The method of claim 18, wherein the duty cycle is maintained below a target value, optionally wherein the target value is 5%, optionally between 1 and 5%, optionally the target value is a value between 2.5% and 7.5%, further optionally the target value is below 10%.

20. The method as claimed in claim 1 further comprising controlling the laser to reduce a pulse repetition rate of the laser, optionally wherein the pulse repetition rate is in the range 0.01 to 5 KHz.

21. The method as claimed in claim 1, wherein the laser output is provided to the sample region without further modulation by a further laser.

22. A semiconductor diode laser spectrometer for measuring radiation absorption by a sample comprising:
a semiconductor diode laser;
an electric pulse generator configured to apply an electrical pulse to the laser thereby to cause the laser to produce a laser output pulse for a gas sample region, wherein the application of the electrical pulse to the semiconductor diode laser causes a change in temperature such that the produced laser output pulse comprises a continuous wavelength chirp over a wavelength range wherein the produced laser output pulse is provided to a gas sample region and wherein at least part of the wavelength range of the wavelength chirp is used as a wavelength scan, wherein the wavelength chirp is caused, at least in part, by the increase in temperature of the laser induced by the applied electrical pulse, and
a detector for detecting optical output from the gas sample region,
wherein the electrical current pulse comprises an electrical pulse length of at least 5 microseconds and/or
wherein the increase in temperature slows over the length of the current pulse thereby causing the rate of change of the continuous wavelength chirp to continuously slow over the length of the pulse from a first initial rate to a second, slower rate, and wherein the detection and/or further sampling of the optical output is performed at the second, slower rate.

23. The spectrometer as claimed in claim 22 wherein at least one of a), b), c):
a) the spectrometer further comprises an optical cell, for example, a non-resonant optical cell, and wherein at least part of the sample region is in the optical cell;
b) the laser comprises an intra-pulse laser spectroscopy system, optionally wherein the intra-pulse spectroscopy is configured to detect compounds using direct absorption;
c) the spectrometer forms part of an open path sensing system, for example, a cross stack system.

24. The spectrometer as claimed in claim 22, wherein the detector comprises a photodetector and/or digitising circuitry, wherein at least one of:
a) the bandwidth of the photodetector is in the range 1 to 10 MHz, optionally higher or lower depending on application;
b) the bandwidth of the digitising circuitry is in the range 1 to 10 MHz, optionally higher or lower depending on application;
c) the detector comprise detection and digitising circuitry having a bandwidth in the range 1 to 20 MHz.
